(19) United States
(12) Patent Application Publication
(10) Pub. No.: US 2010/0114027 A1
(43) Pub. Date: May 6, 2010

(54) FLUID MEDICATION DELIVERY SYSTEMS FOR DELIVERY MONITORING OF SECONDARY MEDICATIONS

(75) Inventors: James D. Jacobson, Lindenhurst, IL (US); Brian Barclay, Pleasant Prairie, WI (US); Marwan A. Fathallah, Mundelein, IL (US); Thomas D. Johnson, Gurnee, IL (US); Michael G. Lowery, Wildwood, IL (US)

Correspondence Address: BRIAN R. WOODWORTH, 275 N. FIELD DRIVE, DEPT. NLEG BLDG H-1, LAKE FOREST, IL, 60045-2579, US

(73) Assignee: HOSPIRA, INC., Lake Forest, IL (US)

(21) Appl. No.: 12/480,989

(22) Filed: Jun. 9, 2009

Related U.S. Application Data
(60) Provisional application No. 61/111,377, filed on Nov. 5, 2008.

(51) Int. Cl. A61M 5/168; A61M 5/142

(52) U.S. Cl. 604/151; 604/253

(57) ABSTRACT

A fluid medication delivery system comprises a primary medication reservoir, a secondary medication reservoir, an infusion pump, a first valve assembly, a second valve assembly, a first y-site, and a fluid flow sensor assembly. The primary medication reservoir has a first fluid. The secondary medication reservoir has a second fluid. The infusion pump pumps fluid from at least one of the primary medication reservoir and the secondary medication reservoir. The first valve assembly controls the flow of fluid from the primary medication reservoir in a first fluid line segment. The second valve assembly controls the flow of fluid from the secondary medication reservoir in a second fluid line segment. The fluid flow sensor assembly determines the flow rate of a fluid from the secondary medication reservoir in the second fluid line segment.

### CROSS REFERENCE TO RELATED APPLICATIONS

This application claims priority under 35 U.S.C. 119 of U.S. Ser. No. 61/111,377 filed Nov. 5, 2008.

### TECHNICAL FIELD

The present invention generally relates to a flow sensor assembly, such as a differential pressure based flow sensor assembly, and method for monitoring medication delivery from a secondary medication reservoir utilizing a system containing the flow sensor assembly, and more particularly to a differential pressure based flow sensor assembly that has a disposable portion and a reusable portion and may be utilized to measure fluid flow from a secondary medication reservoir.

### BACKGROUND

Modern medical devices, including medical pumps, are increasingly being controlled by microprocessor based systems to deliver fluids, solutions, medications, and drugs to patients. A typical control for a medical pump includes a user interface enabling a medical practitioner to enter the dosage of fluid to be delivered, the rate of fluid delivery, the duration, and the volume of a fluid to be infused into a patient. Typically, drug delivery is programmed to occur as a continuous infusion or as a single bolus dose.

It is common for a plurality of medications to be infused to a patient by using a multi-channel infusion pump or using a plurality of single channel infusion pumps where a different fluid is administered from each channel. Another method of delivering multiple medications to a patient is to deliver a first medication using an infusion pump, and additional medications through single bolus doses.

A further common medication delivery system utilizes a primary medication reservoir and a secondary medication reservoir. Medication from the secondary medication reservoir is delivered to a patient after the primary medication reservoir is stopped, such as by clamping the line from the primary reservoir, and resetting the pump to deliver the secondary medication at an appropriate rate for the secondary medication. Once the secondary medication reservoir is empty, the line from the primary medication reservoir is reopened, and medication flows once again from the primary reservoir once the pump is re-programmed to resume the delivery rate appropriate for the first reservoir. This type of system requires a caregiver to manually operate the valves to ensure that flow of medication is coming from the appropriate reservoir, and that the pump is operating at the correct rate for the primary or secondary medication.

However, in other applications involving a secondary medication reservoir, the secondary medication reservoir is simply placed higher than the primary medication reservoir so that the pump draws the medication from the higher secondary medication reservoir until the secondary reservoir is empty, and then flow will resume from the lower primary medication reservoir. Such a system requires that a caregiver carefully monitor the flow to ensure that medication from the appropriate reservoir is being delivered to the patient. In this type of system, it is possible for the wrong medication to be delivered, or the proper medication may be delivered at an inappropriate rate.

Thus, under both previous approaches, a caregiver had to carefully monitor the fluid delivery to ensure that medication being delivered to a patient was coming from the appropriate source, and to further ensure that the medication is being delivered to the patient at the appropriate flow rate. Even with careful oversight from a caregiver, it may be difficult to ensure that the appropriate medication is being delivered to the patient, particularly if the flow rate is low. When the flow rate of the medication is low, a great deal of time may pass prior to the caregiver being able to visually notice a change in volume of medication in a particular reservoir. Also, events where both reservoirs are contributing to the fluid volume drawn through the pump can be very difficult to discern visually. Thus, if medication is being delivered to the patient from an incorrect medication reservoir, a long period of time may pass before corrective action is taken. It is important to confirm that flow has been initiated from the appropriate reservoir and that this reservoir continues to be the active fluid source for as long as desired.

Further, even if the proper medication is being delivered, a caregiver may not be able to discern that the medication is not being delivered at a proper rate. Thus, medication may be delivered too rapidly, or too slowly, and a caregiver may only notice subsequent to a reservoir being empty sooner than planned, or still containing medication when the reservoir should be empty. Thus, a sensor within the flow path from the secondary medication reservoir to the patient, that is capable of measuring flow rate through the flow path, would be helpful to ensure that both the correct medication source is being used, and that the correct amount of the medication is being delivered. Further, it is desirable to provide a robust flow rate sensing methodology that is low cost and in particular introduces low incremental cost to the disposable medication delivery tubing set. Further, it is desirable to provide a flow rate sensing methodology that is capable of accurately sensing the flow rate of fluids that have a range of physical properties, including fluid viscosity, which may not be known precisely. Further, it is desirable to confirm that actual flow from secondary reservoirs is captured and communicated to the caregiver and the electronic medication administration record of the patient in an automated fashion. Further, it is desirable to subject secondary medications to the framework of safety software. Therefore, a need exists for a fluid flow sensor system adapted for monitoring medication delivery.

### SUMMARY

According to one embodiment, a fluid medication delivery system comprises a primary medication reservoir, a secondary medication reservoir, an infusion pump, a first valve assembly, a second valve assembly, a first y-site, and a fluid flow sensor assembly. The primary medication reservoir has a first fluid. The secondary medication reservoir has a second fluid. The infusion pump pumps fluid from at least one of the primary medication reservoir and the secondary medication reservoir. The first valve assembly controls the flow of fluid from the primary medication reservoir in a first fluid line segment. The second valve assembly controls the flow of fluid from the secondary medication reservoir in a second fluid line segment. The fluid flow sensor assembly determines the flow rate of a fluid from the secondary medication reservoir in the second fluid line segment. In one embodiment, the fluid flow sensor assembly is a differential pressure based fluid flow sensor assembly.

According to another embodiment, a fluid medication delivery system comprises a primary medication reservoir, a secondary medication reservoir, an infusion pump, a first valve assembly, a second valve assembly, a first y-site, and a drip counter assembly. The primary medication reservoir has a first fluid. The secondary medication reservoir has a second fluid. The infusion pump pumps fluid from at least one of the primary medication reservoir and the secondary medication reservoir. The first valve assembly controls the flow of fluid from the primary medication reservoir in a first fluid line segment. The second valve assembly controls the flow of fluid from the secondary medication reservoir in a second fluid line segment. The drip counter assembly determines the flow rate of a fluid from the secondary medication reservoir in the second fluid line segment by counting drops of fluid that flow past the drip counter assembly in a portion of the second fluid line segment.

According to a further embodiment, a fluid medication delivery system comprises a primary medication reservoir, a secondary medication reservoir, an infusion pump, a first valve assembly, a second valve assembly, a first y-site, and a load cell assembly. The primary medication reservoir has a first fluid. The secondary medication reservoir has a second fluid. The infusion pump pumps fluid from at least one of the primary medication reservoir and the secondary medication reservoir. The first valve assembly controls the flow of fluid from the primary medication reservoir in a first fluid line segment. The second valve assembly controls the flow of fluid from the secondary medication reservoir in a second fluid line segment. The load cell assembly determines the change in weight of the secondary medication reservoir over time to determine the flow rate of a fluid from the secondary medication reservoir.

According to yet another embodiment, a fluid medication delivery system comprises a medication reservoir, an adjustable valve assembly, and a fluid flow sensor assembly. The medication reservoir has a first fluid. The adjustable valve assembly has a slider to allow for the adjustment of the flow rate of the first fluid through the valve. The second valve assembly controls the flow of fluid from the secondary medication reservoir in a second fluid line segment. The fluid flow sensor assembly determines the flow rate of a fluid from the medication reservoir and has a display to provide a visual indication of the fluid flow rate.

### BRIEF DESCRIPTION OF THE DRAWINGS

FIG. 1 is a pictorial view that illustrates a patient connected to IV line having a flow sensor assembly according to one embodiment;

FIG. 2 shows a closer, more detailed pictorial view of the flow sensor assembly of the embodiment of FIG. 1;

FIG. 3 is an isometric view of the flow sensor assembly of the embodiment of FIG. 1;

FIG. 4 is an isometric cross-sectional view taken along line 4-4 of FIG. 3;

FIGS. 5a-5e illustrate cross-sections of flow restricting elements within differential pressure based flow sensor assemblies according to various embodiments;

FIG. 6 is a pictorial view illustrating delivery of medication to a patient via an IV push or bolus through an IV line having the flow sensor assembly of FIG. 1;

FIG. 7 schematically illustrates a method of delivering medication using a system having a flow sensor assembly according to one basic process;

FIG. 7a schematically illustrates a method of delivering medication using a system with flow sensor assembly, according to a more elaborate process than FIG. 7;

FIGS. 8a-8b schematically illustrate a method of delivering medication using a system having a flow sensor assembly according to another process;

FIG. 9 is a pictorial view that illustrates a medication delivery system having a differential pressure based flow sensor assembly located in a secondary medication reservoir fluid flow path according to one embodiment;

FIG. 10 is a pictorial view that illustrates a medication delivery system having a drip counter sensor located in a secondary medication reservoir fluid flow path according to another embodiment;

FIG. 11 is a pictorial view that illustrates a medication fluid delivery system having a load cell in communication with a secondary medication reservoir according to a further embodiment;

FIG. 12 is a pictorial view that illustrates a medication delivery system having a differential pressure based flow sensor assembly located in a secondary medication reservoir fluid flow path according to yet another embodiment;

FIG. 13 is a pictorial view that illustrates a medication delivery system having a drip counter sensor located in a secondary medication reservoir fluid flow path according to yet a further embodiment;

FIG. 14a is a pictorial view that illustrates a gravity fed medication delivery system having a flow sensor and an adjustable valve according to one embodiment; and

FIG. 14b is a detailed view of the manually adjustable valve shown in FIG. 14a.

### DETAILED DESCRIPTION

While this invention is susceptible of embodiments in many different forms, there is shown in the drawings and will herein be described an example of the invention. The present disclosure is to be considered as an example of the principles of the invention. It is not intended to limit the broad aspect of the invention to the examples illustrated.

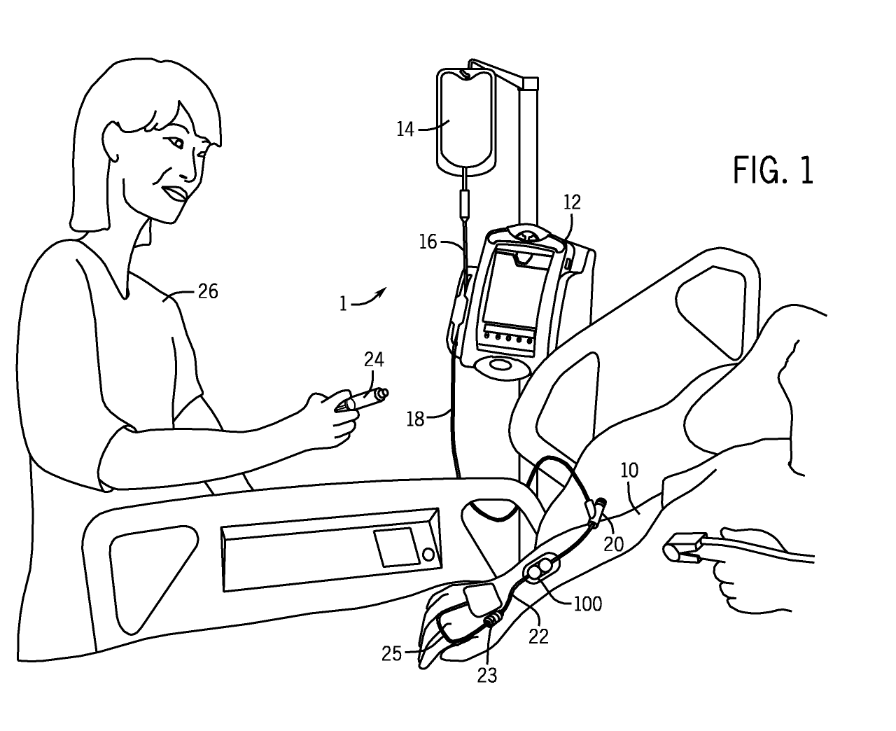

FIG. 1 is a pictorial representation of a patient 10 connected to a medication delivery system 1 and receiving a first medication via an infusion pump 12 from a medication reservoir 14. A first fluid line segment 16 delivers the first medication from the reservoir 14 to the infusion pump 12. The second fluid line segment 18 delivers the medication from the infusion pump 12 to a differential pressure based flow sensor assembly 100. A third fluid line segment 22 delivers the medication from the differential pressure based flow sensor 100 to the patient 10. While three fluid lines segments are described in connection with FIG. 1, it is contemplated that the number of fluid lines or line segments used in connection with the present invention may vary, and may be more or less than three fluid lines. It is further contemplated that fluid lines 16, 18, and 22 can be integrated in manufacturing to present a single common tubing set or line-set. The third fluid line segment 22 is typically connected to the patient 10 through a connector valve 23 and a patient access device such as a catheter 25.

The second fluid line segment 18 has a connection 20 adapted to receive a second medication from a second source. The connection illustrated in FIG. 1 is typically referred to as a Y-Site, although it is contemplated that other connection types and configurations may be used in connection with the present invention.

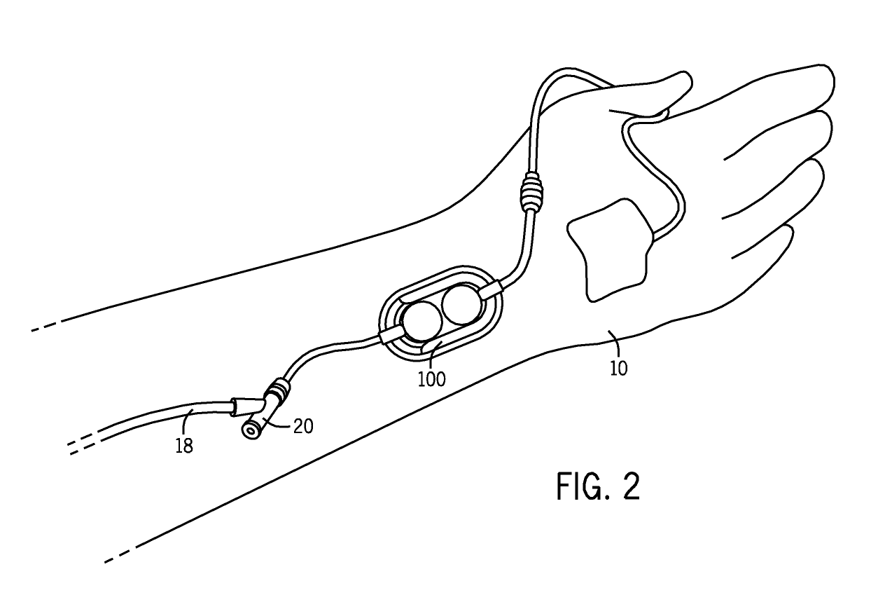

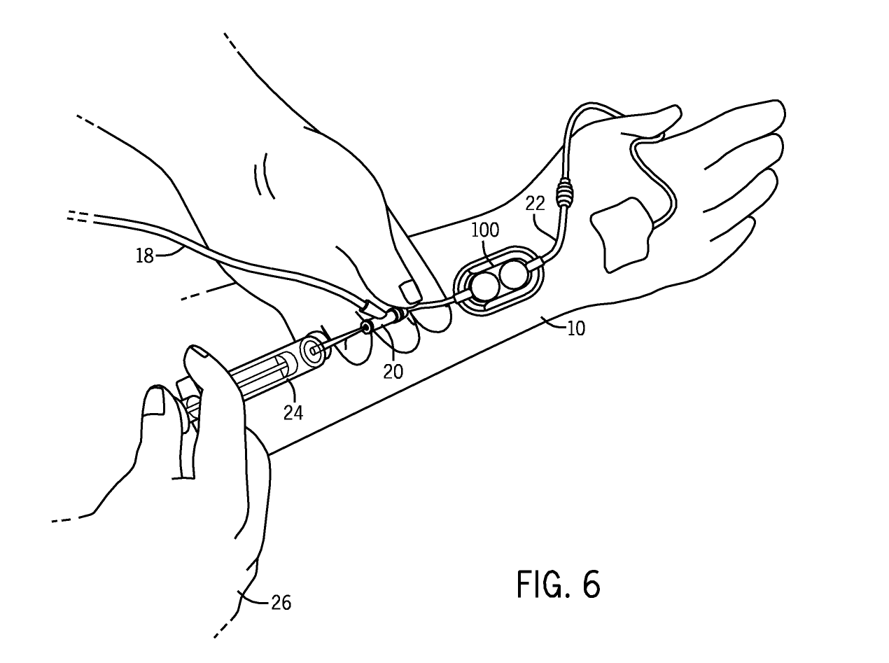

The connection 20, shown in additional detail in FIG. 2, may receive a second medication from a syringe 24 in the form of a manual IV push or bolus by a caregiver 26 (see FIG. 6). It is further contemplated that the second medication may be provided in another fashion, such as from a secondary medication reservoir or other known medication delivery source. The medication delivery system 1 further has a differential pressure based flow sensor assembly 100. In the illustrated embodiment, the differential pressure based flow sensor assembly 100 is located downstream of the connector 20 and is secured on the patient 10. Thus, the flow sensor assembly is adapted to have both the first and the second medication pass through the sensor assembly 100. However, the sensor assembly 100 could also be disposed in any number of locations including but not limited to upstream of the fluid junction between the first and second medication, connected between the second source and the connector 20, or integrally formed on or within one of the branches of the connector 20. The flow sensor assembly 100 need not be secured to the patient 10 directly.

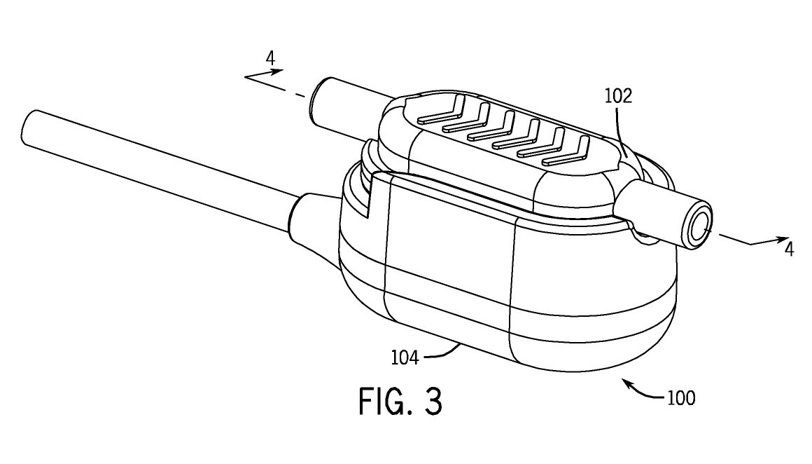

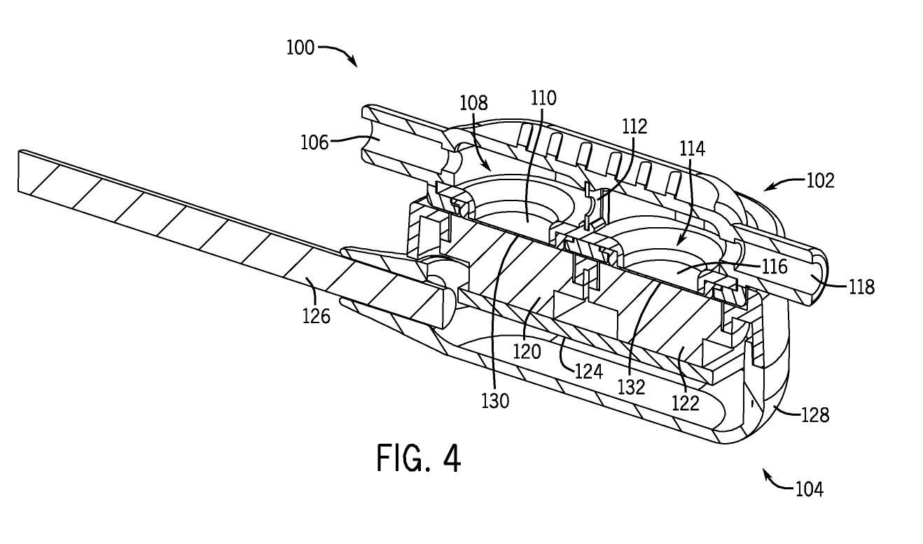

Turning next to FIG. 3 and FIG. 4, the differential pressure based flow sensor assembly 100 is shown in additional detail. The differential pressure based flow sensor assembly 100 has a disposable portion 102 and a reusable portion 104. As used herein reusable is defined as a component that is capable of being safely reused. For example, the same reusable portion 104 can be used multiple times on the same patient with the disposable portion 102 being changed at least every 72 hours or so. The same reusable portion 104 can be used hundreds or even thousands of times on different patients, subject to the cleaning policies recommended by the manufacturer or the healthcare institution, by installing a new disposable portion 102. This is possible since the reusable portion 104 is designed to be robust and to prevent fluid ingress. As may best be seen in FIG. 4, the disposable portion 102 has a fluid inlet 106 an upstream fluid chamber 108, an upstream fluid pressure membrane 110, a flow restricting element 112, a downstream fluid chamber 114, a downstream fluid pressure membrane 116, and a fluid outlet 118. The membranes 110 and 116 are fluid impermeable. Although full membranes are shown, it is contemplated that other types of seals, including but not limited to one or more gaskets and O-rings, would suffice to keep fluid out of the housing of the reusable portion. Any exposed areas could be swabbed with a cleaning solution, if necessary.

As shown in FIG. 4, medication enters the disposable portion 102 through the fluid inlet 106. The medication flows into the upstream fluid chamber 108 from the fluid inlet 106. Next, the medication flows through the flow restricting element 112 and into the downstream fluid chamber 114. The flow of the medication through the flow restricting element 112 results in a drop in fluid pressure as the fluid flows from the upstream fluid chamber 108 to the downstream fluid chamber 114 through the flow restricting element 112. Thus, during forward fluid flow under normal conditions, the fluid pressure within the upstream fluid chamber 108 is generally greater than the fluid pressure within the downstream fluid chamber 114. The fluid pressure within the upstream fluid chamber 108 presses against the upstream fluid pressure membrane 110. Similarly, the fluid pressure within the downstream fluid chamber 114 presses against the downstream fluid pressure membrane 116.

It is contemplated that a variety of materials may be utilized for the manufacture of the disposable portion 102. The disposable portion 102 may comprise a thermoplastic. It is contemplated that the flow restricting element 112 may be made of the same thermoplastic as the rest of the disposable portion 102, or may be a different material than the disposable portion 102. Non-limiting examples of the material that may be utilized to form the flow restricting element 112 include silicon, glass, and medical grade thermoplastics and elastomerics. The fluid pressure membranes 110, 116 may comprise a variety of polymeric or elastomeric materials, such as TPE, or silicone.

It is additionally contemplated that the flow restricting element 112 may be formed integrally with the rest of the disposable portion 10, or the flow restricting element 112 may be a separate component mounted and sealed within the disposable portion 102. In either approach, all fluid passing between the fluid inlet 106 and the fluid outlet 118 is routed through the flow restricting element 112.

As may also be seen in FIG. 4, the reusable portion 104 of the differential pressure based flow rate sensor assembly 100 has an upstream pressure sensor 120, a downstream pressure sensor 122, a circuit board 124, and an electrical connection 126, all contained within a housing 128. The upstream pressure sensor 120 is adapted to interact with the upstream fluid pressure membrane 110 to generate a reading of fluid pressure within the upstream fluid chamber 108. Similarly, the downstream pressure sensor 122 is adapted to interact with the downstream fluid pressure membrane 116 to generate a reading of fluid pressure within the downstream fluid chamber 114. The circuit board 124 receives output from both the upstream pressure sensor 120 and the downstream pressure sensor 122. The circuit board 124 may calculate a pressure difference between the upstream fluid chamber 108 and the downstream fluid chamber 114, or the circuit board 126 may generate an output signal that is transmitted to another device with a processor, such as the infusion pump 12, that calculates the pressure difference between the upstream chamber 108 and the downstream chamber 114. Output of the circuit board 124 passes through electrical connection 126 to the infusion pump 12 (FIG. 1).

Although a wired electrical connection 126 is shown in FIG. 4, the system may optionally comprise wireless electrical connection and communication with the infusion pump 12 or other system components. It is additionally contemplated that according to some alternative embodiments, the reusable portion 104 may further contain additional electronics, such as, batteries, one or more memories, amplifiers, signal conditioning components, analog-to-digital converters, power converters, LED indicators, a display, sound generating components, a wireless communication engine, inductive coils for receiving power from the infusion pump 12 or another source, and active or passive radio frequency identification devices (RFID). It will be appreciated that the calculations and processing described herein can take place on the circuit board 124, in the infusion pump 12, in a remote processor (not shown), or be concentrated in only one of the system components, or distributed among one or more of the system components as needed or desired.

The components of the reusable portion 104 are contained within the housing 128. The housing 128 may be manufactured from a polymeric material such as polycarbonate, polyethylene, polyurethane, polypropylene, acrylic, or other known materials. It is further contemplated that an upstream reusable portion membrane 130 may separate the upstream fluid pressure membrane 110 from the upstream fluid pressure sensor 120. Likewise, a downstream reusable portion membrane 132 may separate the downstream fluid pressure membrane 116 from the downstream fluid pressure sensor 122. It is also contemplated that the upstream reusable portion membrane 130 and the downstream reusable portion membrane 132 can be combined into a single unitary membrane or gasket.

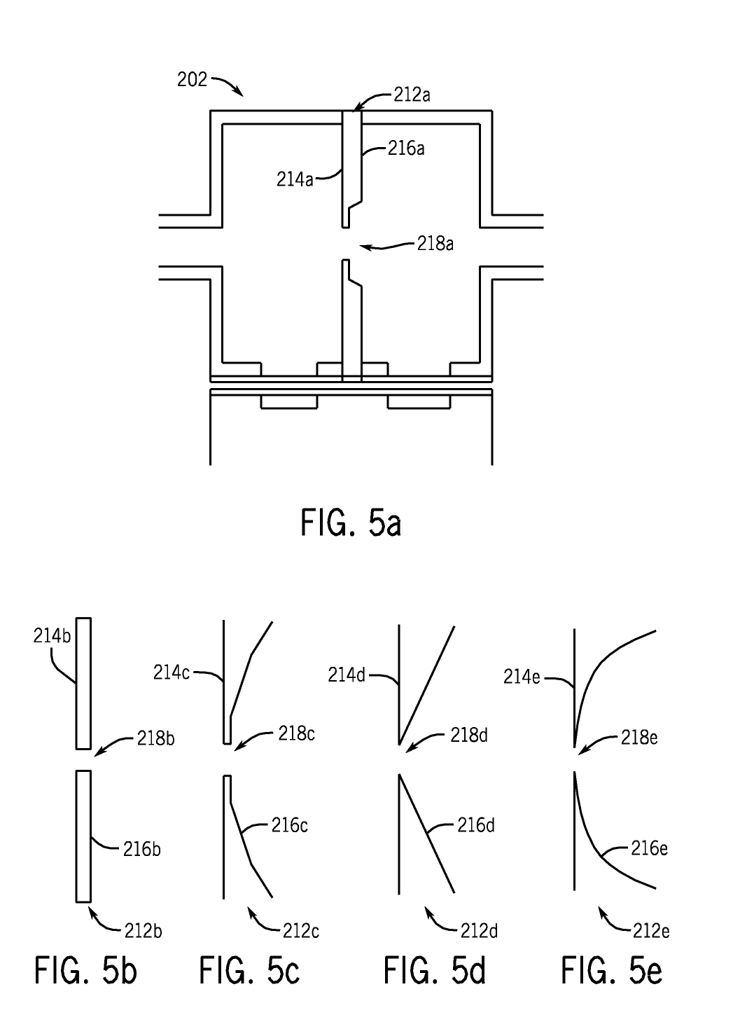

Referring next to FIG. 5a, a cross-section of a disposable portion 202 is schematically illustrated with a flow restricting element 212a to illustrate the profile of the flow restricting element 212a. The flow restricting element 212a may be identical to the flow restricting element 112, but may also vary. The flow restricting element 212a is in the form of an orifice. An orifice may be a beneficial flow restricting element, as orifice performance varies less between fluids of different viscosities than other flow restricting elements, such as capillary channels. That is to say, the measured pressure differential across an orifice for a given flow rate will be largely independent of the viscosity of the active solution, where the pressure difference measured across alternate restrictions such as capillaries will demonstrate a strong dependence upon fluid viscosity. The flow restricting element 212a has a front face 214a located on an upstream side of the flow restricting element 212a, and a rear face 216a on the downstream side of the flow restricting element 212a. An opening 218a is formed through the flow restricting element 212a to allow fluid to flow through the flow restricting element 212a.

The opening 218a may have a variety of aerial shapes, but a circular opening is commonly used as it provides a maximum flow area versus perimeter length. In order to help reduce the effect of fluid viscosity on the flow of the fluid through the opening 218a of the flow restricting element 212a, the opening 218a may have a ratio of a perimeter of the opening 218a to the length the fluid travels though the opening 218a of from about 100:1 to about 2000:1. That is, the perimeter of the opening is sufficiently larger than the length of fluid flow though the opening 218a, such that the pressure drop through the opening 218a is less dependent on the fluid, and more dependent on the geometry of the opening 218a. An opening 218a having a perimeter to flow length ratio of about 1000:1 has been found to be effective. For example, a 430 micron diameter circular orifice with a length in the flow dimension of 12 microns will accommodate flow rates in the hundreds to thousands of ml/hr. A smaller diameter orifice would be needed for smaller flow rates and applications.

The thickness of the opening 218a of the flow restricting element may vary from about 5 microns to about 25 microns. An opening 218a having a thickness of about 12 microns has been found to be effective. In order to demonstrate the desired flow characteristics, it is important to provide a flow orifice or opening in a solid geometry. The ratio of the inlet height, which is to say the minimum internal inlet flow plenum geometry at the orifice plate, to the effective hydraulic diameter of the orifice should be rather large, such as at least 10:4 or about 5:1. However, a constant-thickness membrane, of thickness equal to the length of the desired orifice, may become mechanically weak if the overall area of the membrane is large. Once the orifice opening is established, the membrane material in which the orifice resides can be thicker as one moves away from the orifice perimeter. As a result, the orifice itself can provide the desired restrictive fluid path length, while the membrane in which the orifice resides is thicker than the length of the orifice at a location away from the orifice. Thus, it is contemplated that various other geometries may also be used to form a flow restricting element.

As shown in FIG. 5a, the flow restricting element 212a transitions from a thicker cross sectional shape to a thinner cross sectional shape near the opening 218a. Creating such a geometry for the flow restricting element 212a allows for various low cost manufacturing approaches for the flow restricting element 212a. Creating such a geometry has a limited effect on performance of the flow restricting element 212a, as such a geometry does not introduce a significant pressure difference for fluids having different viscosities, but having the same fluid flow rate. Thus, the thinness of the flow restricting element 212a near the opening 218a limits the effect of fluid viscosity on pressure drop through the opening 218a, while thicker material away from the opening 218a increases the overall strength of the flow restricting element 212a.

FIGS. 5b-5e illustrate alternative flow restricting elements 212b-212e that function similarly to flow restricting element 212a. Flow restricting element 212b maintains a constant thickness, while flow restricting elements 212c-212e are thinner near the openings 218c-218e. Assuming that flow occurs generally from left to right in these figures, the geometry of the rear face 216a-216e does not have a great effect on flow characteristics through openings 218a-218e. This is because flow through the opening 218a-218e typically features well-defined fluid velocity profiles with minimal fluid/wall dynamic interaction on the orifice backside, as long as the rear face 216a-216e geometry is sloped away from the orifice appropriately, and therefore minimizes viscosity induced pressure losses. Some of these orifice geometries lend themselves to manufacturing advantages. For example, orifice 218a can be formed efficiently via silicon processing techniques such as etching, lithography, masking and other MEMS operations. Orifice 218b can be formed efficiently by laser machining thin flat stock material. Orifices 218c and 218d could be formed easily with photo-imaging glass processing techniques. Orifices 218c, 218d, and 218e could be formed using molding or embossing techniques. Further combinations of techniques could be utilized within the scope of the invention.

While many embodiments have been described in connection with an upstream pressure sensor, a flow restricting element, and a downstream pressure sensor within a common assembly, it is further contemplated according to a further alternative embodiment, that these components may be separate standalone components within a fluid flow system. The methods and processes of measuring fluid flow rates and the volume of fluid flow are generally identical to those previously described according to this alternative embodiment. Thus, by monitoring the difference in pressure between a standalone upstream pressure sensor and a standalone downstream pressure sensor generated by fluid flowing through a standalone flow restricting element, the fluid flow rate may be calculated.

Turning next to FIG. 6, an IV push or bolus is shown being delivered to the patient 10. The caregiver 26 connects the syringe 24 to the second fluid line 18 via the connection 20. The caregiver 26 then delivers the mediation within the syringe 24 to the patient through the connection 20. The medication passes through the differential pressure based fluid flow sensor 100 and the third fluid line 22 to the patient 10. The differential pressure based fluid sensor assembly 100 monitors the flow rate of the medication through the sensor assembly 100. By monitoring the flow rate through the sensor assembly 100, the volume of medication delivered to the patient 10 may be calculated.

The flow rate of the fluid through the pressure sensor assembly 100 may be calculated by the following equation:

$$Q = A {C}_{D} \sqrt{\frac{2 Δ P}{ρ}} ,$$

where Q is the volumetric flow rate, ΔP is the pressure differential between an upstream pressure sensor and a downstream pressure sensor, ρ is the fluid mass density, C_{D} is an opening discharge coefficient, and A is the area of the opening. The use of an orifice for the opening has been empirically shown to minimize the dependence of the induced pressure differential on fluid viscosity, and the discharge coefficient remains essentially constant, thus making the flow rate a function of pressure, density, and area.

Once the flow rate Q has been calculated, the volume of the flow may be determined by integrating the flow rate over a period of time using the following equation: V=∫Qdt. Using this equation, both forward and backward flow thorough the sensor assembly 100 may be calculated. A negative flow rate would indicate that the pressure at the downstream sensor 122 is higher than the pressure at the upstream sensor 120, and thus fluid is flowing backwards through the sensor assembly 100, away from the patient 10.

In order to provide a more accurate ΔP, a pressure tare, or calibration of the sensors, may be performed, preferably in a zero flow condition. A pressure tare subtracts the average pressure of both the upstream pressure sensor 120 and the downstream pressure sensor 122 from the readings of the respective upstream and downstream pressure sensors 120, 122 during fluid delivery. Utilizing such a pressure tare reduces the occurrence of signal drifts from pressure supply drifts, amplification, temperature variance, or residual pressures from any priming steps prior to delivering and recording a bolus dose.

Reverse flow of fluid through the sensor can be also measured with ΔP being negative. In this case, the flow is computed by taking the absolute value of ΔP and moving the negative sign outside the square root,

$$Q = - A {C}_{D} \sqrt{\frac{2 \vert Δ P \vert}{ρ}} .$$

Negative flow rates are important to aggregate in the computation of true net forward volume delivery from the syringe, as they may impact the accuracy of total net volume delivered from the syringe. Additionally, an occlusion condition (i.e., the catheter 25 or the patient's vein being closed or occluded) can be detected using a back draw of the syringe prior to forward fluid delivery, a typical clinical practice. Under normal conditions, reverse flow of the fluid can be directly measured and aggregated into the net forward volume delivery. However, under occlusion scenarios, the occluded reverse flow can be quickly detected by the sensor using threshold negative limits of the downstream and upstream sensors drawing a negative vacuum pressure.

The outputs of the upstream pressure sensor 120 and the downstream pressure sensor 122 may further be monitored for detection of motion artifacts to distinguish such artifacts from true flow patterns. To detect motion artifacts, a ratio of the upstream pressure sensor 120 output to the downstream pressure sensor 122 output is monitored. If, for example, the ratio is less than a predetermined threshold, such as 3:1, it is likely that any changes in pressure indicated by the upstream pressure sensor 120 and the downstream pressure sensor 122 are the results of motion artifacts within the sensor assembly 100, not forward fluid flow. Thus, flow is only indicated when the ratio of the pressures indicated by the upstream pressure sensor 120 and the downstream pressure sensor 122 is greater than a threshold amount. This is because once flow is initiated, the flow restricting element 112 causes the pressure at the upstream pressure sensor 120 to be significantly higher than the pressure at the downstream pressure sensor 122. Alternatively, reverse fluid flow is similarly distinguished from motion artifacts, if the ratio of the downstream pressure sensor to the upstream pressure sensor is less than a limit threshold, such as 3:1, and otherwise the signal is considered motion artifacts. Pressure values obtained due to motion artifacts may be excluded from the flow rates and aggregate volume computation. Motion artifacts events are also distinguished from events indicating the true onset of flow, which is used to gate or determine the start of bolus delivery via the syringe 24.

Algorithms also are contemplated to detect the start and end of a single bolus dose. Such an algorithm may rely on a first derivative and a short term mean value of the flow rate. If the mean value of the flow rate is above a certain threshold, such as for example 300 ml/hr, and the mean value of the derivative of the flow rate is above another threshold value, such as 50 (ml/hr)/sec, this flow rate and flow rate derivative indicate a start of a bolus dose. The threshold values are selected based upon the finding that typical bolus dose deliveries have a flow rate between about 300 ml/hr to about 5000 ml/hr, while a human injecting a bolus dose is typically incapable of delivering the injection at a rate less than about 50 ml/hr, on a per second basis.

The outputs of the differential pressure sensor assembly 100 may also be used to monitor both the delivery of medication via a single bolus dose, and via an infusion pump. Such an algorithm would indicate that a flow rate below a threshold level, such as for example 300 ml/hr, is not from a bolus dose. Similarly, infusion pump cycles provide a consistent sinusoidal pattern of deliveries with every pumping cycle. Utilizing an approach that analyzes the output of the sensor assembly 100 in a frequency domain, such as through a Fourier transform, pump infusion cycles appear at a much higher frequency than flow rates introduced through a single bolus dose. A low pass filter with a cutoff frequency separating the frequency band due to an infusion pump action, versus manual delivery via a single bolus dose, can segregate the flow rate signal due to each source. Alternatively, an inverse Fourier transform of the frequencies in the band below the frequencies affected by the pump action can recover a time domain flow rate signal from the differential pressure based sensor assembly 100 to quantify the amount of flow from a single bolus dose. Such an algorithm to isolate flow due to a pump source from flow due to manual injection could also be utilized to verify an infusion pump flow rate. Similarly, pressure pulsations occurring as a result of arterial pulsations when the sensor is in direct fluidic connection with an arterial vessel can be detected and mathematically compensated for using frequency domain low pass filtering below a cutoff frequency, since manual injections are usually lower frequency than arterial pulsations. Alternatively, linear weighted averaging of pressure values measured at the sensor is a form of filtering or smoothing that can be applied on the signal to reduce the effect of pulsations. Typical infusion pumps do not measure flow volume, but rather estimate flow volume based upon pump fluidic displacement. Thus, a differential pressure based flow sensor assembly 100 may verify infusion pump function, or be used in a closed feedback loop to control pump flow rate.

Yet another algorithm contemplated allows the differential pressure based sensor assembly 100 to be used to detect air pockets within fluids flowing through the sensor assembly 100. An air pocket typically is much less dense than a fluid passing through the sensor assembly 100. Thus, an air pocket or bubble within a fluid medium generates an abrupt change in pressure value, followed by a return to expected levels. The start and end of the abrupt change in pressure values is detected by monitoring the first derivative and the second derivative of the output of the upstream pressure sensor 120 and the downstream pressure sensor 122. An abrupt change in pressure would first be noticed on the upstream pressure sensor 120, followed by an abrupt change in pressure on the downstream pressure sensor 122. These pressure changes would be followed by an abrupt resumption back to pressure levels prior to air pocket reception, once the air pocket is passed. The duration of the deviation from typical pressures is indicative of the size of the air pocket.

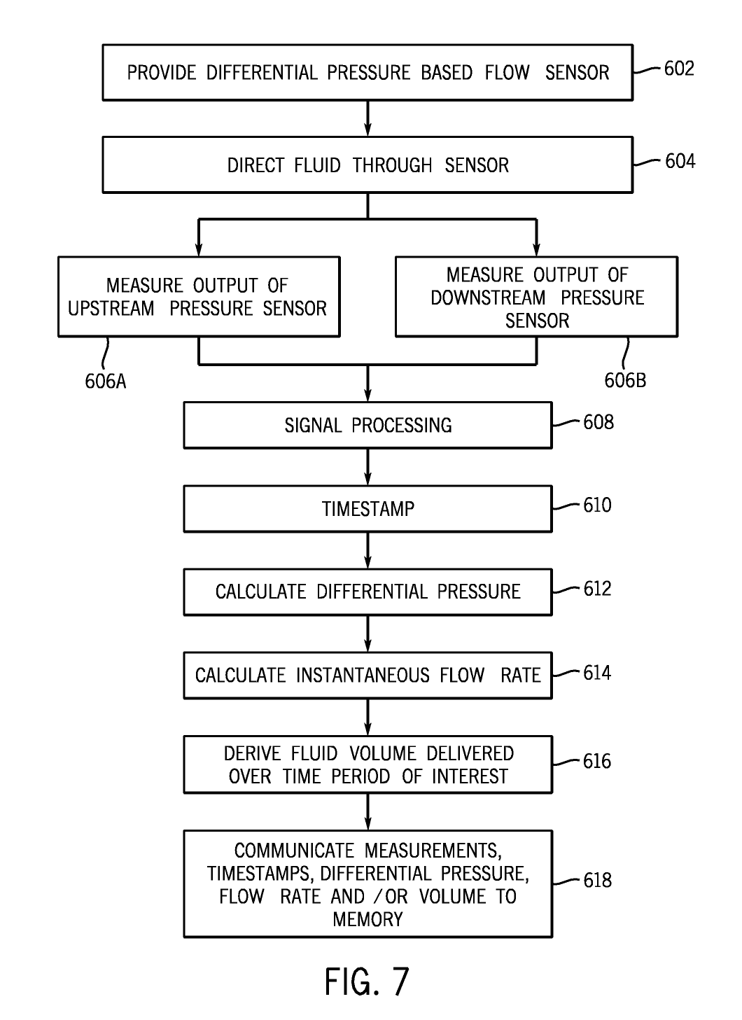

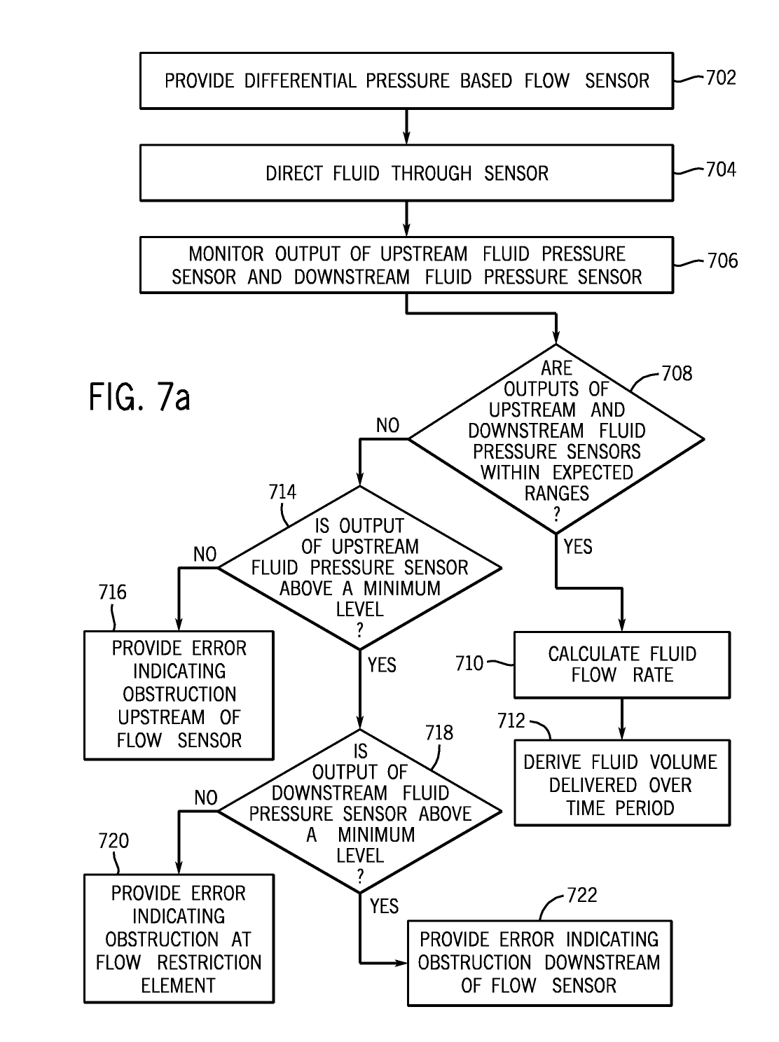

FIG. 7 shows a basic process of utilizing a differential pressure based sensor assembly 100 to determine the instantaneous flow rate and/or volume of a fluid flow delivered through a bolus or other delivery. The process provides a differential pressure based flow sensor assembly 100 in step 602. Fluid flows through the sensor assembly in step 604. The output of the upstream pressure sensor 120 is measured in step 606A, and the output of the downstream pressure sensor 122 is measured in step 606B. The signals from the sensors 120, 122 can be filtered, amplified, or otherwise processed (for example as described above) in step 608. A timestamp is associated with the measurements in step 610. A differential pressure is calculated based upon the observed measurements in step 612. The instantaneous fluid flow rate is calculated in step 614. The flow rate is integrated over time to derive the volume deliver during the time period of interest in step 616. In step 618, the sensor signals or measurements, timestamp information, differential pressure, flow rate and/or volume delivered are communicated to a memory, which can be located in the sensor assembly 100, in the infusion pump 12, or another computer.

Turning now to FIG. 7a, a process of utilizing a differential pressure based sensor assembly to deliver a fluid is depicted, including monitoring for possible occlusions within the delivery system. The process provides a differential pressure based flow sensor in step 702. Fluid flows through the sensor in step 704 and the output of both the upstream fluid pressure sensor and the downstream fluid pressure sensor are monitored in step 706. The process determines whether the outputs of both the upstream fluid pressure sensor and the downstream fluid pressure sensor are within expected ranges in step 708. If so, the process calculates the fluid flow rate, utilizing the algorithm previously described, in step 710. Once the flow rate has been determined, the process derives the volume that has passed through the sensor assembly 100 over a given period of time in step 712. As described above with respect to FIG. 7, the sensor signals or measurements, timestamp information, differential pressure, flow rate and/or volume delivered are communicated to a memory, which can be located in the sensor assembly 100, in the infusion pump 12, or another processor.

If the outputs of the upstream and downstream fluid pressure sensors do not fall within expected ranges, the process determines if the output of the upstream fluid pressure sensor is above a minimum level in step 714. If the pressure is not above a preset minimum level, an error signal is generated in step 716, indicating that a possible obstruction exists upstream of the differential pressure based flow sensor assembly 100. However, if the output of the upstream fluid pressure sensor is above a minimum level, the process in step 718 determines if the output level of the downstream fluid pressure sensor is above a preset minimum level. If the output of the downstream fluid pressure sensor is not above a preset minimum level, an error signal is generated in step 720 that indicates an obstruction may be present at the flow restricting element 112. However, if the downstream fluid pressure sensor detects a pressure above the preset minimum level, an error signal is generated in step 722 indicating that an obstruction may be present downstream of the differential pressure based flow sensor assembly 100.

Thus, utilizing the process illustrated in FIG. 7a, the flow rate of a fluid as well as the volume of the fluid delivered through a differential pressure based flow sensor assembly may be calculated, and an error message may be provided when an occlusion occurs.

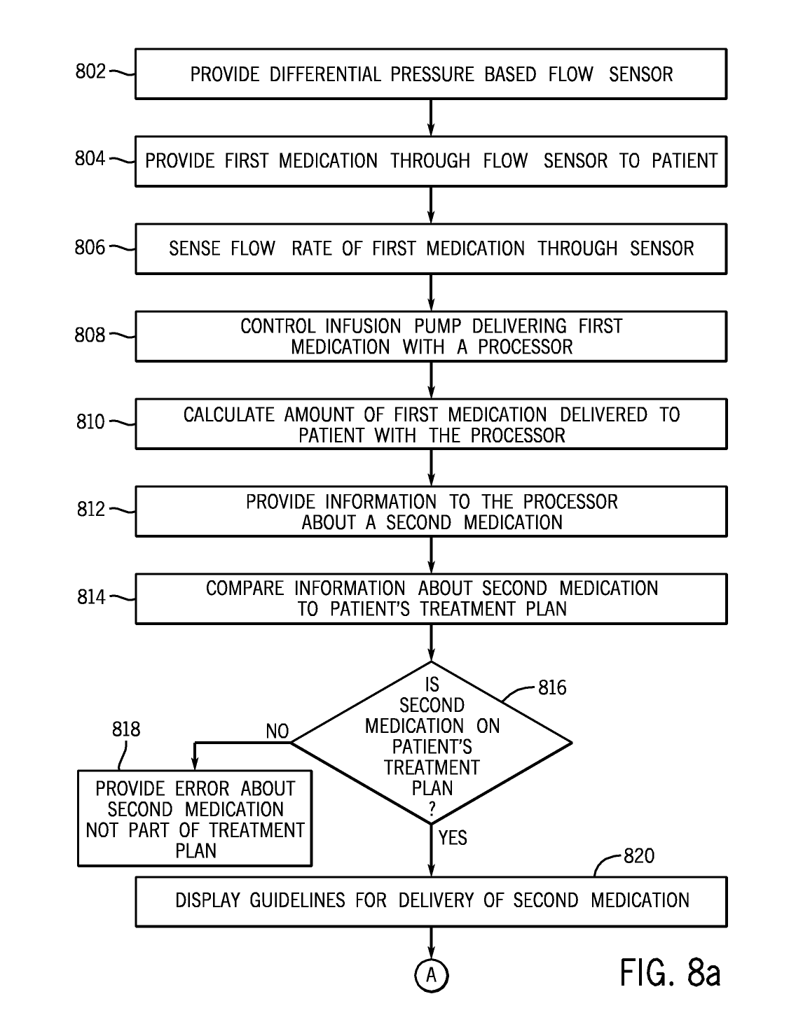

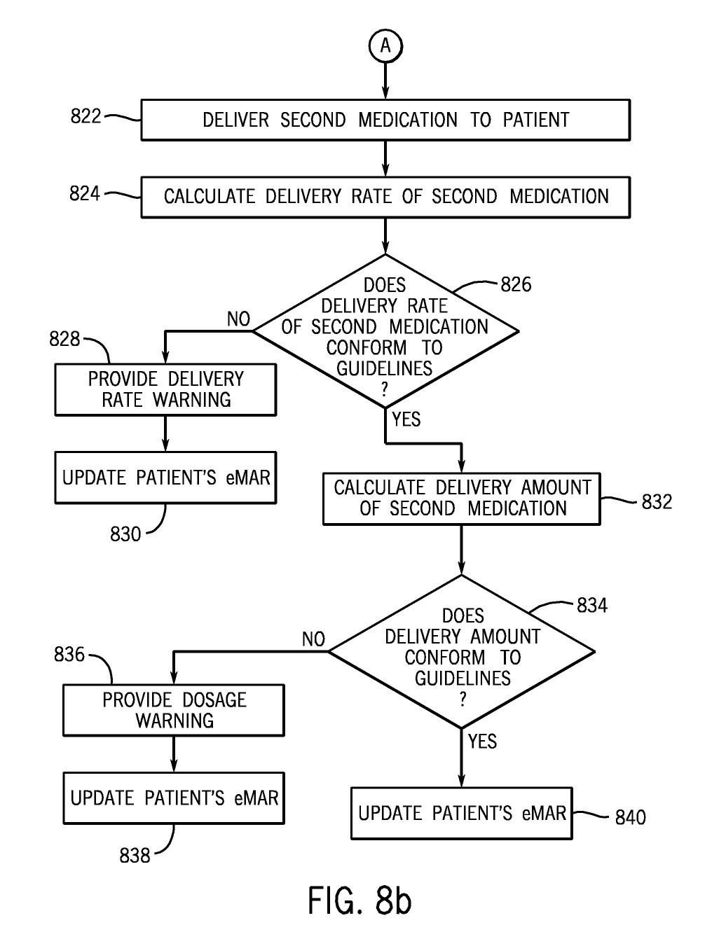

As shown in FIGS. 8a-8b, a method of delivering medication to a patient utilizing a medication delivery system having an infusion pump is depicted in block diagram form. The process provides a differential pressure based flow sensor assembly in step 802, such as sensor assembly 100 previously described herein. A first medication is provided through the flow sensor assembly to the patient 10 in step 804. The flow through the sensor assembly is sensed in step 806. In step 808, the process controls an infusion pump delivering the first medication via a processor. The amount or volume of the first medication delivered to the patient is calculated in step 810 using the processor and signals received from the differential pressure based flow sensor assembly 100. Information about a second medication to be delivered to the patient is provided to the processor in step 812. The information provided about the second medication is compared to information within the patent's treatment plan in step 814. The process determines in step 816 whether the second medication is on the patient's specific treatment plan, such as by checking whether the patient has a medical order or prescription for the second medication. If the second medication is not found on the patient's treatment plan, an error message is provided in step 818 indicating that the second medication is not found on the patient's treatment plan, and the caregiver should check with a physician or other caregiver to determine if it is appropriate to provide the second medication to the patient. It is contemplated that the system may allow the caregiver to override the warning and deliver the second medication. Such an override could be set by the hospital, or other healthcare facility, so as to allow some caregivers to deliver certain medications to a patient even if that medication is not found on the patient's treatment plan. Thus, a balance may be reached between providing a patient a potentially important medication dose, with protecting the patient from the delivery of an unnecessary medication. If the second medication is found on the patient's treatment plan, guidelines for delivering the second medication are generated or displayed in step 820. The guidelines can include but are not limited to a target delivery rate with upper and/or lower limits, a total volume or amount to be delivered during the bolus, and a time period over which to deliver the IV push or bolus.

Continuing now to FIG. 8b, the second medication is delivered to the patient in step 822. The process calculates the delivery rate of the second medication using the differential pressure based flow rate sensor assembly 100 in step 824. As described with respect to FIG. 7 above, the delivery flow rate calculations can be stored in memory. A comparison is performed in step 826 to determine if the delivery rate of the second medication conforms to the delivery guidelines. If the delivery rate does not conform to the delivery guidelines, a delivery rate warning is provided to the caregiver in step 828. If the delivery rate warning is provided, the patient's electronic medication administration record (eMAR) is updated in step 830 to show that the second medication was delivered at a rate inconsistent with the delivery guidelines or protocols. The amount of the second medication delivered to the patient can also be calculated in step 832. The process in step 834 compares the amount of the second medication delivered to the amount of the second medication the patient was scheduled to receive. If the amount of the second medication the patient received does not conform to the patient's treatment plan, a dosage warning is provided to the caregiver at step 836. This warning can indicate that the patient was provided an underdose of the second medication, or that the patient was provided with an overdose of the second medication. The patient's electronic medication administration record (eMAR) is updated in step 838 to include the amount of the second medication that was provided to the patient, as well as information to indicate that the dosage of the second medication did not conform to the patient's treatment plan. If the amount of the second medication delivered to the patient conforms to the patient specific guidelines, the patient's electronic medication administration record (eMAR) is updated in step 840 to indicate that a proper dosage of the second medication was delivered to the patient. It is contemplated that every update to the patient's electronic medication administration record (eMAR) will note the time a medication was delivered to the patient, as well as the caregiver responsible for delivering that medication to the patient.

According to a further embodiment, a disposable infusion tubing set is provided that has a disposable portion of a differential pressure based flow sensor assembly. The tubing set would include at least a first tube adapted to connect to a primary medication reservoir, and a connection site to allow a second medication to be introduced into the first tube of the tubing set upstream of the disposable portion of the differential pressure based flow sensor assembly. The disposable infusion tubing set further has a second tube adapted to connect to a patient access device. The second tube is adapted to be positioned downstream of the disposable portion of the differential pressure based flow sensor assembly. As discussed above, the disposable portion of the differential pressure based flow sensor assembly can be disposed in other locations within the disposable infusion tubing set, depending on the line pressure conditions, delivery flow rates, or fluid volume delivery amounts of interest.

According to yet another embodiment, a differential pressure based flow rate sensor assembly is replaced by a pressure based event detection sensor. A pressure based event detection sensor allows an event, such as a bolus, to be detected noting a spike in pressure. Such an event detection sensor would not allow the computation of the volume of medication delivered, but will place a notation onto a patient's record that some medication was delivered at a specific time. Thus, a record will exist confirming that a patient was provided with medication.

According to yet a further embodiment, a differential pressure based flow sensor assembly may be powered by an inductive power source. Such an embodiment would contain many of the same features as the differential pressure based flow sensor assembly 100 described herein. Similarly, it is contemplated that a wireless differential pressure based flow sensor assembly may transmit information regarding a pressure at an upstream pressure sensor and information regarding a downstream pressure sensor to other components within a system. Finally, it is contemplated that the portion 104 of the differential pressure based flow sensor assembly 100 could be produced using MEMS, integrated circuits or other technology in a miniaturized and low cost manner, such that the portion 104 might be considered disposable as well.

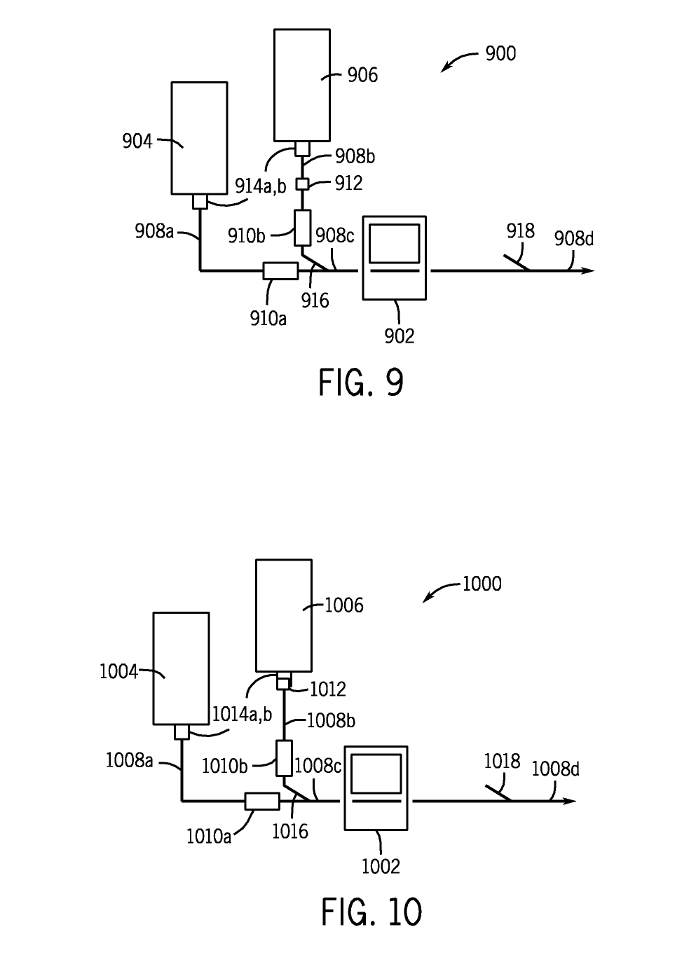

Turning now to FIG. 9, a medication delivery system 900 is shown having an infusion pump 902, a primary medication reservoir 904 and a secondary medication reservoir 906. The medication delivery system 900 allows a patient to receive medication from the primary medication reservoir 904, or the secondary medication reservoir 906, through various fluid line portions 908a-908d depending on the settings of a first valve 910a or a second valve 910b. A first drip chamber 914a and a second drip chamber 914b are in fluid communication with each of the respective primary and secondary medication reservoirs 904, 906. A first fluid line segment 908a delivers a first medication from primary medication reservoir 904 through the first valve 910a to a y-site 916. The first valve 910a allows the flow of the first medication in the first fluid line segment 908a to be controlled.

A second fluid line segment 908b delivers a second medication from a secondary medication reservoir 906 to the y-site 916. The second fluid line segment 908b causes fluid to pass through a differential pressure based fluid flow sensor assembly 912, such as the sensor assembly 100 described above. The second fluid line segment 908b additionally has the second valve 910b, to allow flow through the second line segment to be controlled.

The first fluid line segment 908a and the second fluid line segment 908b fluidly join together at the y-site 916. A third fluid line segment 908c provides a fluid path from the y-site 916 to the pump 902. The pump 902 may be generally identical to the pump 12 described above. The pump 902 may be controlled by the caregiver to deliver medication at a predetermined flow rate.

A fourth fluid line segment 908d delivers fluid from the pump 902 to the patient. The fourth fluid flow path 908d has a second y-site 918 to allow another fluid line or fluid source, such as a syringe bolus, to connect to the fourth fluid line segment 908d and be supplied to the patient.

In use a first medication, or other fluid, in the primary medication reservoir 904 is delivered to the patient via the first fluid line segment 908a, the third fluid line segment 908c, the pump 902 and the fourth fluid line segment 908d. The pump 902 will monitor and control the flow rate as well as the volume of the first medication that passes through the pump 902.

When a fluid from the secondary medication reservoir 906 is to be delivered to the patient, the pump 902 is stopped, and the first valve 910a is closed. The second valve 910b is opened and the pump is reprogrammed, after which fluid flows from the secondary medication reservoir 906 to the patient. The fluid from secondary medication reservoir 906 flows at a predetermined rate based on the characteristics of the second fluid, and the patient's clinical needs, through the fluid flow sensor assembly 912. The fluid flow sensor assembly 912 may be generally identical to the differential pressure based flow sensor assembly 100 described above, or may be a different type of flow sensor assembly. The fluid flow sensor assembly 912 allows the flow rate of the fluid from the secondary medication reservoir 906 to be monitored, and thus, the volume of fluid delivered may be calculated. Further, this flow rate calculations can be compared to the known pump rate and provide confirmation that substantially all of the fluid routing through the pump is in fact originating from secondary reservoir 906.

Once the fluid flow sensor assembly 912 has sensed that the proper amount of fluid from the secondary medication reservoir 906 has been delivered, or the secondary medication reservoir 906 is about to become empty, the caregiver will stop the pump 902, close the second valve 910b, and reopen the first valve 910a to allow the medication in the primary medication reservoir 904 to again be delivered to the patient. The fluid flow sensor assembly 912 allows data monitored by the sensor to be communicated electronically to a patient's electronic medication administration record, such that the patient's medical records accurately reflect when a patient was given the second medication, the flow rate of the delivery of the second medication, and the volume of the second medication that was delivered to the patient. Updating the patient's electronic medication administration record in such a manner helps to prevent errors in medication delivery, by reducing the likelihood that a patient has received a medication that is not indicated in the patient's medical records, or conversely, has not received a medication that the patient's medical record shows was delivered.

In addition to simply monitoring the flow rate of medication from the secondary medication reservoir 906, the fluid flow sensor assembly 912 may also be set to alert the caregiver to changes in flow conditions that may indicate that the caregiver needs to take some action. For instance, if a differential pressure based fluid flow sensor is used for the fluid flow sensor assembly 912 and the pressure of the upstream chamber drops below a predetermined level, the caregiver may be alerted that the fluid level in the secondary medication reservoir 906 is becoming low. Further, monitoring of the flow conditions of medication from the secondary medication reservoir 906 allow the caregiver to be alerted if the flow rate differs from an expected flow rate, such as if an occlusion is present in the fluid line.

Turning next to FIG. 10, a medication delivery system 1000 according to a further embodiment is shown. The medication delivery system 1000 is shown having an infusion pump 1002, a primary medication reservoir 1004 and a secondary medication reservoir 1006. The medication delivery system 1000 allows a patient to receive medication from the primary medication reservoir 1004, or the secondary medication reservoir 1006, through various fluid line portions 1008a-1008d, depending on the settings of a first valve 1010a or a second valve 1010b. A first drip chamber 1014a and a second drip chamber 1014b are in fluid communication with each of the respective primary and secondary medication reservoirs 1004, 1006. A first fluid line segment 1008a delivers a first medication from primary medication reservoir 1004 through the first valve 1010a to a y-site 1016. The first valve 1010a allows the flow of the first medication in the first fluid line segment 1008a to be controlled.

A second fluid line segment 1008b delivers a second medication from a secondary medication reservoir 1006 to the y-site 1016. The second fluid line segment 1008b causes fluid to pass through a drop counter sensor assembly 1012 that is adapted to count each drop of fluid from the secondary medication reservoir 1006 that enters the second fluid line portion 1008b.

According to one embodiment, the drop counter sensor assembly 1012 estimates the flow rate by assuming each fluid drop has a predetermined volume, thus, based on the number of fluid drops that pass the sensor over a given period, a flow rate may be calculated. According to a different embodiment, it is contemplated that the drop counter assembly 1012 calculates the flow rate by estimating the volume of each fluid drop that passes by the drop counter assembly 1012. By estimating the volume of each fluid drop, a more accurate fluid flow rate may be calculated, as some variation commonly occurs in the size of the fluid drops.

The second fluid line segment 1008b additionally has the second valve 1010b, to allow flow through the second line segment 1008b to be controlled.

The first fluid line segment 1008a and the second fluid line segment 1008b fluidly join together at the y-site 1016. A third fluid line segment 1008c provides a fluid path from the y-site 1016 to the pump 1002. The pump 1002 may be generally identical to the pump 12 described above. The pump 1002 may be controlled by the caregiver to deliver medication at a predetermined flow rate.

A fourth fluid line segment 1008d delivers fluid from the pump 1002 to the patient. The fourth fluid flow path 1008d has a second y-site 1018 to allow another fluid line or fluid source, such as a syringe bolus, to connect to the fourth fluid line segment 1008d and be supplied to the patient.

In use a first medication, or other fluid, in the primary medication reservoir 1004 is delivered to the patient via the first fluid line segment 1008a, the third fluid line segment 1008c, the pump 1002 and the fourth fluid line segment 1008d. The pump 1002 will monitor the flow rate as well as the volume of the first medication that passes through the pump 1002.

When a fluid from the secondary medication reservoir 1006 is to be delivered to the patient, the pumping operation of the pump 1002 is ceased, and the first valve 1010a is closed. The second valve 1010b is opened, and subsequent to reprogramming the pump 1002, fluid flows from the secondary medication reservoir 1006 to the patient at the appropriate secondary rate. The fluid from secondary medication reservoir 1006 flows through the drop counter assembly 1012. The drop counter sensor assembly 1012 allows the flow rate of the fluid from the secondary medication reservoir 1006 to be monitored, and thus, the volume of fluid delivered may be calculated. The flow rate observed by the drop counter sensor assembly 1012 can be compared to the anticipated flow rate controlled by the pump 1002, thus allowing the system 1000 to confirm that substantially all of the fluid progressing through the pump 1002 originates within the secondary reservoir 1006. In the event that the system 1000 detects that the pump 1002 is drawing fluid from a source other than the secondary reservoir 1006 the caregiver may be notified, or the pump 1002 may cease pumping operations, as appropriate based on the medication involved and the healthcare facility or hospital policy.

Once the drop counter sensor assembly 1012 has sensed that the proper amount of fluid from the secondary medication reservoir 1006 has been delivered, or the secondary medication reservoir 1006 is about to become empty, the caregiver will stop the pump 1002, close the second valve 1010b, and reopen the first valve 1010a to allow the medication in the primary medication reservoir 1004 to again be delivered to the patient subsequent to a reprogramming of the pump infusion rate. The drop counter sensor assembly 1012 allows data monitored by the sensor to be communicated electronically to a patient's electronic medication administration record, such that the patient's medical records accurately reflect when a patient was given the second medication, the flow rate of the delivery of the second medication, and the volume of the second medication that was delivered to the patient. Updating the patient's electronic medication administration record in such a manner helps to prevent errors in medication delivery, by reducing the likelihood that a patient has received a medication that is not indicated in the patient's medical records, or conversely, has not received a medication that the patient's medical record shows was delivered.

In addition to simply monitoring the flow rate of medication from the secondary medication reservoir 1006, the fluid flow sensor assembly 1012 may also be set to alert the caregiver to changes in flow conditions that may indicate that the caregiver needs to take some action. These alerts could include reductions in flow from the reservoir, which could indicate a near-empty reservoir state, or a mode in which the pump is drawing from both reservoirs.

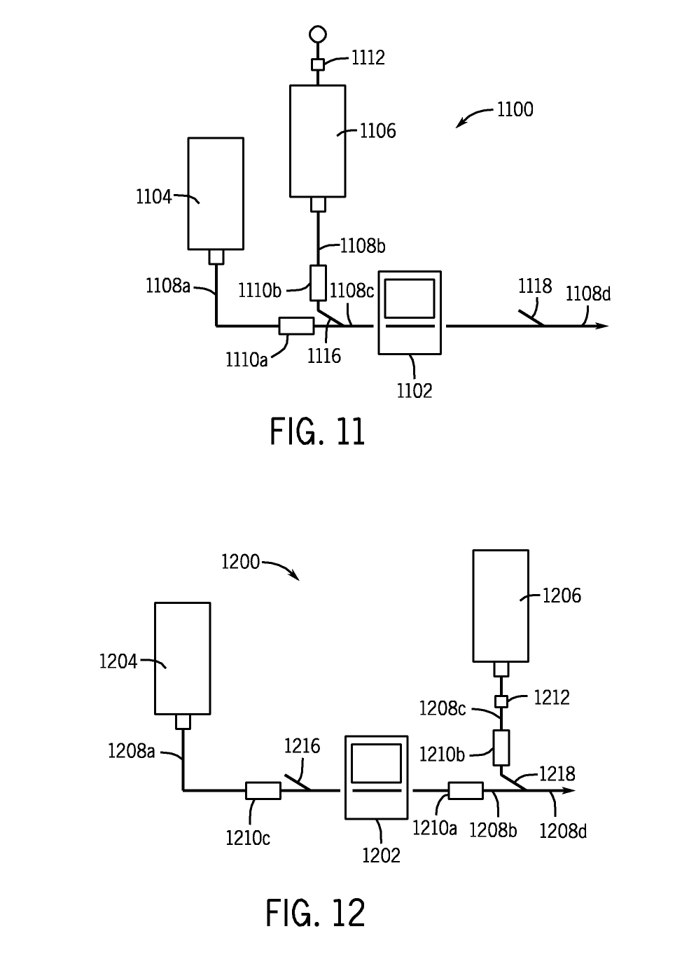

As shown in FIG. 11, a further embodiment of a medication delivery system 1100 is depicted. The medication delivery system 1100 is shown having an infusion pump 1102, a primary medication reservoir 1104 and a secondary medication reservoir 1106. The medication delivery system 1100 allows a patient to receive medication from the primary medication reservoir 1104, or the secondary medication reservoir 1106, through various fluid line portions 1108a-1108d depending on the settings of a first valve 1010a or a second valve 1110b. A first fluid line segment 1108a delivers a first medication from the primary medication reservoir 1104 through the first valve 1110a to a y-site 1116. The first valve 1110a allows the flow of the first medication in the first fluid line segment 1108a to be controlled.

A second fluid line segment 1108b delivers a second medication from a secondary medication reservoir 1106 to the y-site 1116. The secondary medication reservoir 1106 is connected to a load cell 1112. The load cell 1112 is adapted to measure the weight of the secondary medication reservoir 1106. The load cell 1112 is adapted to record the weight of the secondary medication reservoir 1106 over time, thus allowing the flow rate of fluid out of the secondary medication reservoir 1106 to be calculated, by monitoring the change in weight over time of the secondary medication reservoir 1106 and dividing that result by the density of the fluid within the secondary medication reservoir 1106. Further, the total volume of fluid delivered may be calculated by dividing the total change in weight by the density of the fluid.

The second fluid line segment 1108b causes fluid to pass through the second valve 1110b to allow the flow of the fluid from the secondary reservoir 1106 to be controlled.

The first fluid line segment 1108a and the second fluid line segment 1108b fluidly join together at the first y-site 1116. A third fluid line segment 1108c provides a fluid path from the first y-site 1116 to the pump 1102. The pump 1102 may be generally identical to the pump 12 described above. The pump 1102 may be controlled by the caregiver to deliver medication at a predetermined flow rate.

A fourth fluid line segment 1108d delivers fluid from the pump 1102 to the patient. The fourth fluid flow path 1108d has a second y-site 1118 to allow another fluid line or fluid source, such as a syringe bolus, to connect to the fourth fluid line segment 1108d and be supplied to the patient.

In use a first medication, or other fluid, in the primary medication reservoir 1104 is delivered to the patient via the first fluid line segment 1108a, the third fluid line segment 1108c, the pump 1102 and the fourth fluid line segment 1108d. The pump 1102 will monitor and control the flow rate as well as the volume of the first medication that passes through the pump 1102.

When a fluid from the secondary medication reservoir 1106 is to be delivered to the patient, the pump 1102 is stopped, and the first valve 1110a is closed. The second valve 1110b is opened and fluid flows from the secondary medication reservoir 1106 to the patient, subsequent to reprogramming of the pump 1102. The load cell 1112 allows the flow rate of the fluid from the secondary medication reservoir 1106 to be monitored, and thus, the volume of fluid delivered may be calculated. Additionally, when the secondary medication reservoir 1106 is positioned upstream of the pump 1102, the output of the load cell 1112 may be compared to the flow rate calculated by the pump 1102 to ensure that only medication from the secondary reservoir 1106 is being delivered to the patient.

While fluid is being delivered from the secondary medication reservoir 1106, the pump 1102 is restarted, and pumps the fluid from the secondary medication reservoir 1106 at a predetermined rate based on the characteristics of the second fluid, and the patient's clinical needs.

Once the load cell 1112 has indicated that the proper amount of fluid from the secondary medication reservoir 1106 has been delivered, or the secondary medication reservoir 1106 is about to become empty, the care giver may stop the pump 1102, close the second valve 1110b, and reopen the first valve 1110a to allow the medication in the primary medication reservoir 1104 to again be delivered to the patient. The load cell 1112 allows data monitored by the sensor to be communicated electronically to a patient's electronic medication administration record, such that the patient's medical records accurately reflect when a patient was given the second medication, the flow rate of the delivery of the second medication, and the volume of the second medication that was delivered to the patient.

In addition to simply monitoring the flow rate of medication from the secondary medication reservoir 1106, the load cell 1112 may also be set to alert the caregiver to changes in flow conditions that may indicate that the caregiver needs to take some action.

Yet another embodiment of a medication delivery system 1200 is depicted in FIG. 12. The medication delivery system 1200 generally identical to the medication delivery system 900 depicted in FIG. 9, except the secondary medication reservoir connects to the fluid line portion at the second y-site 1218 downstream of an infusion pump 1202. The medication delivery system 1200 is shown having the infusion pump 1202, a primary medication reservoir 1204 and a secondary medication reservoir 1206. The medication delivery system 1200 allows a patient to receive medication from the primary medication reservoir 1204, or the secondary medication reservoir 1206, through various fluid line portions 1208a-1208d depending on the settings of a first valve 1210c, a second valve 1210b, or a third valve 1210c. A first fluid line segment 1208a delivers a first medication from the primary medication reservoir 1204 through the third valve 1210c to a y-site 1216. The third valve 1210c allows the flow of the first medication in the first fluid line segment 1208a to be controlled. It is contemplated that the first valve 1210a may not be required if the pump 1202 has check valves to limit the backflow in the second fluid line segment 1208b when fluid flows from the secondary medication reservoir 1206. Additionally, the pump 1202 itself may sufficiently limit backflow, even if the pump 1202 does not contain check valves, to allow the elimination of the first valve 1210a.

A second fluid line segment 1208b runs from the infusion pump 1202 to the second y-site 1218. The second fluid line segment 1208b contains the first valve 1210a that allows the flow of fluid in the second fluid line segment 1208b to be controlled.

The third fluid line segment 1208c delivers a second medication from a secondary medication reservoir 1206 to a second y-site 1218. The third fluid line segment 1208c causes fluid to pass through a differential pressure based fluid flow sensor assembly 1212, such as the sensor assembly 100 described above. The third fluid line segment 1208c additionally has the second valve 1210b, to allow flow through the third line segment 1208c to be controlled. The second valve 1210b may be a proportional or analog valve to allow the caregiver to vary the rate of the fluid flow from the secondary reservoir 1206.

The second fluid segment 1208b and the third fluid line segment 1208c fluidly join together at the second y-site 1218. A fourth fluid line segment 1208d delivers fluid from the second y-site 1218 to the patient.

In use, a first medication, or other fluid, in the primary medication reservoir 1204 is delivered to the patient via the first fluid line segment 1208a, the second fluid line segment 1208b, the pump 1202 and the fourth fluid line segment 1208d. The pump 1202 will monitor and control the flow rate as well as the volume of the first medication that passes through the pump 1202.

When a fluid from the secondary medication reservoir 1206 is to be delivered to the patient, the pump 1202 is stopped, and the first valve 1210a and the third valve 1210c are closed. The second valve 1210b is opened, and fluid flows from the secondary medication reservoir 1206 to the patient. The fluid from secondary medication reservoir 1206 flows through the fluid flow sensor assembly 1212. The fluid flow sensor assembly 1212 may be generally identical to the differential pressure based flow sensor assembly 100 described above, or may be a different type of flow sensor assembly. The fluid flow sensor assembly 1212 allows the flow rate of the fluid from the secondary medication reservoir 1206 to be monitored, and thus, the volume of fluid delivered may be calculated. Flow rate information derived by the sensor can be communicated via a user interface, including a user interface on the pump 1202.

While fluid is being delivered from the secondary medication reservoir 1206, the pump 1202 remains stopped, and gravity feeds the fluid from the secondary medication reservoir 1206 to the patient. Alternatively, a pressure cuff may be applied to the secondary bag to increase the infusion rate. This is particularly useful in treatments requiring high continuous flow rates of delivery. During the initiation of flow from the secondary medication reservoir 1206, the flow sensor 1212 output may be monitored and the valve 1210b adjusted to provide an appropriate fluid flow from the secondary reservoir 1206.

Once the fluid flow sensor assembly 1212 has sensed that the proper amount of fluid from the secondary medication reservoir 1206 has been delivered, or the secondary medication reservoir 1206 is about to become empty, the care giver may close the second valve 1210b, and reopen the first valve 1210a and the third valve 1210c to allow the medication in the primary medication reservoir 1204 to again be delivered to the patient, subsequent to re-initiation of the infusion pump 1202. The fluid flow sensor assembly 1212 allows data monitored by the sensor to be communicated electronically to a patient's electronic medication administration record, such that the patient's medical records accurately reflect when a patient was given the second medication, the flow rate of the delivery of the second medication, and the volume of the second medication that was delivered to the patient. Updating the patient's electronic medication administration record in such a manner helps to prevent errors in medication delivery, by reducing the likelihood that a patient has received a medication that is not indicated in the patient's medical records, or conversely, has not received a medication that the patient's medical record shows was delivered.

In addition to simply monitoring the flow rate of medication from the secondary medication reservoir 1206, the fluid flow sensor assembly 1212 may also be set to alert the caregiver to changes in flow conditions that may indicate that the caregiver needs to take some action. For instance, if a differential pressure based fluid flow sensor is used for the fluid flow sensor assembly 1212 and the pressure of the upstream chamber drops below a predetermined level, the caregiver may be alerted that the fluid level in the secondary medication reservoir 1206 is becoming low. Further, monitoring of the flow conditions of medication from the secondary medication reservoir 1206 allow the caregiver to be alerted if the flow rate differs from an expected flow rate, such as if an occlusion is present in the fluid line.

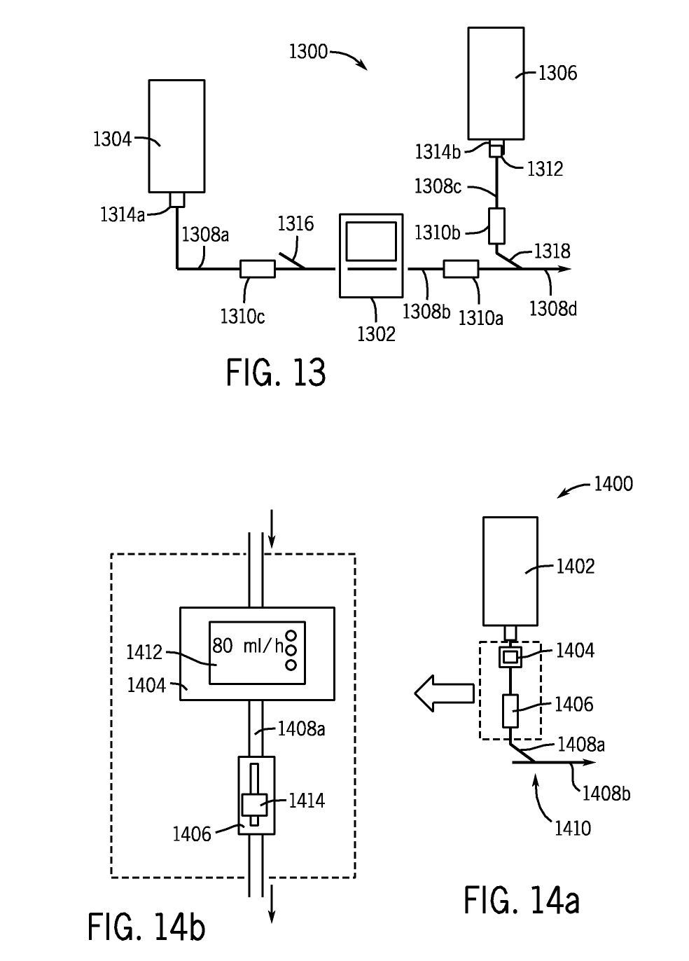

Referring now to FIG. 13, yet a further embodiment of a medication delivery system 1300 is depicted. The medication delivery system 1300 is generally identical to the medication delivery system 1000, except the secondary fluid reservoir connects to the system downstream of the infusion pump 1302.

The medication delivery system 1300 is shown having an infusion pump 1302, a primary medication reservoir 1304 and a secondary medication reservoir 1306. The medication delivery system 1300 allows a patient to receive medication from the primary medication reservoir 1304, or the secondary medication reservoir 1306, through various fluid line portions 1308a-1308d depending on the settings of a first valve 1310a, a second valve 1310b, and a third valve 1310c. A first drip chamber 1314a and a second drip chamber 1314b are in fluid communication with each of the respective primary and secondary medication reservoirs 1304, 1306. A first fluid line segment 1308a delivers a first medication from primary medication reservoir 1304 through the third valve 1310c to a first y-site 1316 and finally to the infusion pump 1302. The third valve 1310c allows the flow of the first medication in the first fluid line segment 1308a to be controlled.

A second fluid line segment 1308b runs from the infusion pump 1302 to the second y-site 1318.

A third fluid line segment 1308c delivers the second medication from the secondary medication reservoir 1306 to the second y-site 1318. The third fluid line segment 1308c causes fluid to pass through a drop counter sensor assembly 1312 that is adapted to count each drop of fluid from the secondary medication reservoir 1306 that enters the third fluid line portion 1308c.

According to one embodiment, the drop counter sensor assembly 1312 estimates the flow rate by assuming each fluid drop has a predetermined volume, thus, based on the number of fluid drops that pass the sensor over a given period, a flow rate may be calculated. According to a different embodiment, it is contemplated that the drop counter assembly 1312 calculates the flow rate by estimating the volume of each fluid drop that passes by the drop counter assembly 1312. By estimating the volume of each fluid drop, a more accurate fluid flow rate may be calculated, as some variation commonly occurs in the size of the fluid drops.

The third fluid line segment 1308c additionally has the second valve 1310b, to allow flow through the third line segment 1308c to be controlled.

The second fluid line segment 1308b and the third fluid line segment 1308c fluidly join together at the second y-site 1318. The pump 1302 may be generally identical to the pump 12 described above. The pump 1302 may be controlled by the caregiver to deliver medication at a predetermined flow rate.

The fourth fluid line segment 1308d delivers fluid from the second y-site 1318 to the patient.

In use, a first medication, or other fluid, in the primary medication reservoir 1304 is delivered to the patient via the first fluid line segment 1308a, the pump 1302, the second fluid line segment 1308b, and the fourth fluid line segment 1308d. The pump 1302 will monitor and control the flow rate as well as the volume of the first medication that passes through the pump 1302.

When a fluid from the secondary medication reservoir 1306 is to be delivered to the patient, the pump 1302 is stopped, and the first valve 1310a and the third valve 1310c are closed. The second valve 1310b is opened, and fluid flows from the secondary medication reservoir 1306 to the patient. The fluid from secondary medication reservoir 1306 flows through the drop counter assembly 1312. The drop counter sensor assembly 1312 allows the flow rate of the fluid from the secondary medication reservoir 1306 to be monitored, and thus, the volume of fluid delivered may be calculated.

While fluid is being delivered from the secondary medication reservoir 1306, the pump 1302 remains off, and gravity causes the fluid flow from the secondary medication reservoir 1306. Alternatively, a pressurized cuff may be used to increase the pressure driving the fluid from the secondary medication reservoir 1306. This is particularly useful in treatments requiring high continuous flow rates for delivery of fluid from the secondary medication reservoir 1306. During the initiation of the flow of fluid from the secondary medication reservoir 1306, the flow sensor 1312 output may be monitored to allow the caregiver to adjust the valve 1310b to provide appropriate fluid flow.

Once the drop counter sensor assembly 1312 has sensed that the proper amount of fluid from the secondary medication reservoir 1306 has been delivered, or the secondary medication reservoir 1306 is about to become empty, the caregiver closes the second valve 1310b, and reopens the first valve 1310a and the third valve 1310c to allow the medication in the primary medication reservoir 1304 to again be delivered to the patient subsequent to re-initiation of the infusion pump 1302. The drop counter sensor assembly 1312 allows data monitored by the sensor to be communicated electronically to a patient's electronic medication administration record, such that the patient's medical records accurately reflect when a patient was given the second medication, the flow rate of the delivery of the second medication, and the volume of the second medication that was delivered to the patient. Updating the patient's electronic medication administration record in such a manner helps to prevent errors in medication delivery, by reducing the likelihood that a patient has received a medication that is not indicated in the patient's medical records, or conversely, has not received a medication that the patient's medical record shows was delivered.

In addition to simply monitoring the flow rate of medication from the secondary medication reservoir 1306, the fluid flow sensor assembly 1312 may also be set to alert the caregiver to changes in flow conditions that may indicate that the caregiver needs to take some action.

Finally, turning to FIGS. 14a and 14b, still yet another embodiment of a medication delivery system 1400 is depicted. As shown in FIG. 14a, the medication delivery system has a fluid reservoir 1402, a flow sensor assembly 1404, that may be a differential pressure based flow sensor assembly operationally similar to the flow sensor 100 described above, an adjustable control valve 1406, and fluid line portions 1408a and 1408b. An optional y-site 1410 fluidly joins a first fluid line portion 1408a to a second fluid line portion 1408b. The y-site 1410 allows another fluid line or fluid source, such as a syringe bolus to connect to the second fluid line segment 1408b to be supplied to the patient.

In use, a medication, or other fluid, in the reservoir 1402 is delivered to the patient via the first fluid line segment 1408a and the second fluid line segment 1408b. The adjustable valve 1406 allows the flow rate of the fluid to be adjusted based on the clinical needs of the patient. As shown in FIG. 14b, the flow sensor assembly 1404 additionally has a display 1412. The display may show a real time flow rate as determined using the sensor assembly 1404. By observing the displayed flow rate in real time on the display 1412, the caregiver may adjust the position of a slider 1414 of the adjustable control valve 1416 to either increase or decrease the flow rate. Thus, the medication delivery system 1400 allows a gravity fed medication delivery to occur with a flow rate based on a patient's clinical needs. For example, if the display 1412 shows that the flow rate is higher than desired, the caregiver may adjust the valve slider 1414 to partially close the valve 1406 until the display 1412 indicates the desired flow rate. Similarly, if the display 1412 indicates that the flow rate is lower than desired, the care giver may adjust the valve slider 1414 to partially open the valve 1406 until the display 1412 indicates the desired flow rate.

Once the fluid flow sensor assembly 1404 has sensed that the proper amount of fluid from the reservoir 1402 has been delivered, or the reservoir 1402 is about to become empty, the caregiver may close the adjustable valve 1406. Depending on the patient's medical needs, the caregiver may then replace the reservoir 1402 with another reservoir, or may simply remove the empty reservoir 1402. The fluid flow sensor assembly 1404 allows data monitored by the sensor to be communicated electronically to a patient's electronic medication administration record, such that the patient's medical records accurately reflect when a patient was given the medication, the flow rate of the delivery of the medication, and the volume of the medication that was delivered to the patient. Updating the patient's electronic medication administration record in such a manner helps to prevent errors in medication delivery, by reducing the likelihood that a patient has received a medication that is in not indicated in the patient's medical records, or conversely, has not received a medication that the patient's medical record shows was delivered. While infusion data from an infusion pump may easily be captured and communicated to caregivers and electronic patient records, the present embodiments outline cost-effective and practical techniques by which to capture medication delivery data not subject to pump based infusions.

It should be noted that the systems may not require or utilize the manual liquid valves described above, as a pump may draw fluid from whichever reservoir is physically positioned at a higher elevation. In such a mode of operation, a flow sensor provides similar information that allows the source of the fluid flow to be identified.

While the foregoing has described what is considered to be the best mode and/or other examples, it is understood that various modifications may be made and that the subject matter disclosed herein may be implemented in various forms and examples, and that they may be applied in numerous other applications, combinations and environments, only some of which have been described herein. Those of ordinary skill in that art will recognize that the disclosed aspects may be altered or amended without departing from the true scope of the subject matter. Therefore, the subject matter is not limited to the specific details, exhibits and illustrated examples in this description. It is intended to protect any and all modifications and variations that fall within the true scope of the advantageous concepts disclosed herein.

We claim:

1. A fluid medication delivery system comprising:
a primary medication reservoir having a first fluid;
a secondary medication reservoir having a second fluid;
an infusion pump to pump fluid from at least one of the primary medication reservoir and the secondary medication reservoir;
a first valve assembly adapted to control the flow of fluid from the primary medication reservoir in a first fluid line segment;
a second valve assembly adapted to control the flow of fluid from the secondary medication reservoir in a second fluid line segment;
a first y-site joining the first fluid line segment and the second fluid line segment; and
a fluid flow sensor assembly adapted to automatically determine the flow rate of a fluid from the secondary medication reservoir in the second fluid line segment.

2. The fluid medication delivery system of claim 1, wherein the flow sensor is a differential pressure based fluid flow sensor that comprises:
a disposable portion having:
a body defining a fluid flow passage forming an inlet and an outlet;
a flow restricting element positioned along the fluid flow passage between the inlet and the outlet;
an upstream fluid pressure membrane at a location in the fluid flow passage between the inlet and the flow restricting element; and
a downstream fluid pressure membrane at a location in the fluid flow passage between the flow restricting element and the outlet; and
a reusable portion having:
an upstream fluid pressure sensor to sense an upstream fluid pressure at an upstream location in the fluid flow passage between the inlet and the flow restricting element, the upstream fluid pressure sensor being positioned to generally determine the fluid pressure at the upstream fluid pressure membrane; and
a downstream fluid pressure sensor to sense a downstream fluid pressure at a downstream location in the fluid flow passage between the flow restricting element and the outlet, the downstream fluid pressure sensor being positioned to generally determine the fluid pressure at the downstream fluid pressure membrane.

3. The fluid medication delivery system of claim 1, wherein the y-site is located at an upstream location of the infusion pump.

4. The fluid medication delivery system of claim 3, wherein the infusion pump is adapted to pump fluid from both the primary medication reservoir and the secondary medication reservoir.

5. The fluid medication delivery system of claim 4, wherein the first valve assembly is in an open position and the second valve assembly is in a closed position when the first fluid is being delivered to the patient.

6. The fluid medication delivery system of claim 4, wherein the first valve assembly is in a closed position and the second valve assembly is in an open position when the second fluid is being delivered to the patient.

7. The fluid medication delivery system of claim 1, wherein the y-site is located at a downstream location of the infusion pump.

8. The fluid medication delivery system of claim 7, wherein the infusion pump is adapted to pump fluid from only the primary medication reservoir.

9. The fluid medication delivery system of claim 7, wherein fluid from the secondary medication delivery system is gravity fed.

10. The fluid medication delivery system of claim 1, wherein information from the fluid flow sensor is added to a patient's electronic medication administration record.

11. A fluid medication delivery system comprising:
a primary medication reservoir having a first fluid;
a secondary medication reservoir having a second fluid;
an infusion pump to pump fluid from at least one of the primary medication reservoir and the secondary medication reservoir;
a first valve assembly adapted to control the flow of fluid from the primary medication reservoir in a first fluid line segment;
a second valve assembly adapted to control the flow of fluid from the secondary medication reservoir in a second fluid line segment;
a first y-site joining the first fluid line segment and the second fluid line segment;
a drip counter assembly adapted to determine the flow rate of a fluid from the secondary medication reservoir by counting the number of drops that pass the drip counter assembly in a portion of the second fluid line segment.

12. The fluid medication delivery system of claim 11, wherein the drip counter assembly determines the fluid flow rate based on estimating the volume of each fluid drop counted.

13. The fluid medication delivery system of claim 11, wherein the drip counter assembly determines the fluid flow rate based on a predefined volume for each fluid drop counted.

14. The fluid medication delivery system of claim 11, wherein the y-site is located at an upstream location of the infusion pump.

15. The fluid medication delivery system of claim 11, wherein the y-site is located at a downstream location of the infusion pump.

16. The fluid medication delivery system of claim 15, wherein the infusion pump is adapted to pump fluid from only the primary medication reservoir.

17. The fluid medication delivery system of claim 11, wherein information from the fluid drip counter assembly is added to a patient's electronic medication administration record.

18. A fluid medication delivery system comprising:
a primary medication reservoir having a first fluid;
a secondary medication reservoir having a second fluid;
an infusion pump to pump fluid from at least one of the primary medication reservoir and the secondary medication reservoir;
a first valve adapted to control the flow of fluid from the primary medication reservoir in a first fluid line segment;
a second valve assembly adapted to control the flow of fluid from the secondary medication reservoir in a second fluid line segment;
a first y-site joining the first fluid line segment and the second fluid line segment; and
a load cell assembly adapted to determine the flow rate of a fluid from the secondary medication reservoir by determining the change in weight over time of the secondary medication reservoir.

19. The fluid medication delivery assembly of claim 18, wherein the load cell assembly determines the fluid flow rate based on the density of the second fluid.

20. The fluid medication delivery system of claim 18, wherein the y-site is located at an upstream location of the infusion pump.

21. The fluid medication delivery system of claim 18, wherein the y-site is located at a downstream location of the infusion pump.

22. The fluid medication delivery system of claim 21, wherein the infusion pump is adapted to pump fluid from only the primary medication reservoir.

23. The fluid medication delivery system of claim 18, wherein information from the load cell assembly is added to a patient's electronic medication administration record.

24. A fluid medication delivery system comprising:
a medication reservoir having a first fluid;
a flow sensor assembly adapted to determine the flow rate of the first fluid from the medication reservoir, the flow sensor assembly having a display for providing a visual indication of the fluid flow rate; and
an adjustable valve assembly having an adjustment mechanism to allow for the adjustment of the flow rate of the first fluid through the valve.

25. The fluid medication delivery system of claim 24, wherein the flow sensor assembly is a differential pressure based fluid flow sensor that comprises:
a disposable portion having:
a body defining a fluid flow passage forming an inlet and an outlet;
a flow restricting element positioned along the fluid flow passage between the inlet and the outlet;
an upstream fluid pressure membrane at a location in the fluid flow passage between the inlet and the flow restricting element; and
a downstream fluid pressure membrane at a location in the fluid flow passage between the flow restricting element and the outlet; and
a reusable portion having:
the display to provide a visual indication of the fluid flow rate;
an upstream fluid pressure sensor to sense an upstream fluid pressure at an upstream location in the fluid flow passage between the inlet and the flow restricting element, the upstream fluid pressure sensor being positioned to generally determine the fluid pressure at the upstream fluid pressure membrane; and
a downstream fluid pressure sensor to sense a downstream fluid pressure at a downstream location in the fluid flow passage between the flow restricting element and the outlet, the downstream fluid pressure sensor being positioned to generally determine the fluid pressure at the downstream fluid pressure membrane.

26. The fluid medication delivery system of claim 24, wherein the display provides a real-time flow rate.

27. The fluid medication delivery system of claim 24, wherein information from the differential pressure based fluid flow sensor is added to a patient's electronic medication administration record.

28. The fluid medication delivery system of claim 24, wherein the adjustment mechanism of the adjustable valve assembly is a slider.

29. The fluid medication delivery system of claim 24, wherein the display provides a cumulative total of the volume of fluid delivered.
